(19) United States
(12) Patent Application Publication
(10) Pub. No.: US 2026/0069880 A1
(43) Pub. Date: Mar. 12, 2026

(54) MULTI -MODAL ENVIRONMENTALLY CONTROLLABLE CHAMBER

(71) Applicant: Visibelle Derma Institute, Inc., Bloomington, MN (US)

(72) Inventor: Steven J. Daffer, Edina, MN (US)

(21) Appl. No.: 19/325,764

(22) Filed: Sep. 11, 2025

Related U.S. Application Data
(60) Provisional application No. 63/693,256, filed on Sep. 11, 2024.

(51) Int. Cl. A61N 5/06; A61M 11/00

(52) U.S. Cl. CPC A61N 5/06; A61M 11/00; A61M 2202/0007; A61M 2202/0208; A61M 2202/04; A61M 2205/052; A61M 2205/3334; A61M 2205/3344; A61M 2205/3379; A61M 2205/502; A61N 2005/0627; A61N 2005/0639; A61N 2005/0652; A61N 2005/0659

(57) ABSTRACT

A therapeutic device includes a base having a first plurality of light emitting devices and a bed supported by the base. A hood is movable between an open position and a closed position, wherein the hood comprises a second plurality of light emitting devices. The device includes first and second end caps attached to opposing ends of the base, wherein when the hood is in the closed position, a substantially enclosed chamber is formed between the base, the hood and the end caps. The device includes at least one airborne energy configured to be delivered into the enclosed chamber through at least one of the end caps. A controller is configured to control the type, intensity and duration of light emitted from the first and second plurality of light emitting devices and the at least one airborne energy to control an environment within the enclosed chamber.

### CROSS-REFERENCE TO RELATED APPLICATION

The present application is based on and claims the benefit of U.S. provisional patent application Ser. No. 63/693,256, filed Sep. 11, 2024, the content of which is hereby incorporated by reference in its entirety.

### BACKGROUND

The present disclosure relates to an environmentally controllable chamber that emits at least light in the visible and/or infrared spectrum along with a plurality of modes that supply different energies within the chamber. More particularly, the present disclosure relates to an environmentally controllable chamber in which a person lies to receive light therapy and other energies.

Subjecting the human body to different wavelengths of visible light and/or infrared light at adequate intensities provides health benefits to the user. Many people desire to utilize photonic energy for treatments or to enhance the effects of exercise, physical therapy, massage and the like.

The different photonic energies provide different benefits to the user. By way of non-limiting example, red light (620-750 nm) in the visible light spectrum is known to improve skin appearance, such as reducing wrinkles, scars, redness and acne and aid in recovery after working out or experiencing trauma to the person's body. Green light (525-550 nm) is known to improve the appearance of the skin by lessening dark circles, pigmentation, broken capillaries and sunspots and calm irritated or over-stimulated skin. Blue light therapy (405-410 nm) can be used to treat acne and can improve skin tone and texture. Yellow light therapy (580-590 nm) can reduce the appearance of solar lentigos. Full spectrum white light (400-700 nm) can uplift mood and enhance meditative calm.

The photonic energies also include those in the infrared spectrum (800-1200 nm) which can be used for detoxification, pain relief, reduction of muscle tension, relaxation, improved circulation, weight loss, skin purification, boosting of the immune system, aid in lowering of blood pressure and aid in recovery from physical exercise. The photonic energies also include those in the ultraviolet spectrum (280-380 nm) which can be used to treat skin disorders such as psoriasis, vitiligo, destroy bacteria and promote wound healing.

In order to provide the benefits of photonic energies to a user, a hinged tubular structure, similar to that of a tanning bed, can be equipped with visible light emitting devices that emit light along the length of the tube. The user gains entrance to the tubular structure by lifting a hood hingedly attached to a base and laying on the base. With the user laying on the base, the hood is lowered to surround the user's body so that the user's body is subjected to the light therapy. However, the tubular structure has open ends that does not allow for the environment within the tubular structure to be controlled.

There is a need to control the environment within the tubular structure to provide different energies along with the light therapy to enhance to effectiveness of the light therapy and/or to synergistically cooperate with the light therapy to modify the outcome of the light therapy.

### SUMMARY

An aspect of the present disclosure relates to a therapeutic device having a base with a length and a first plurality of light emitting devices disposed in the base substantially along the length. A bed is within the base and is configured to support a user, where the bed is substantially optically transparent to allow light therethrough. A hood is movably attached to the base and is movable between an open position and a closed position, wherein the hood comprises a second plurality of light emitting devices. The device includes first and second end caps attached to opposing ends of the base, wherein when the hood is in the closed position, a substantially enclosed chamber is formed between the base, the hood and the end caps. The device includes at least one airborne energy configured to be delivered into the enclosed chamber through at least one of the end caps. A controller is configured to control the type, intensity and duration of light emitted from the first and second plurality of light emitting devices and the at least one airborne energy to control an environment within the enclosed chamber.

Another aspect of the present disclosure relates to a therapeutic device having a base with a length and a first plurality of light emitting devices disposed in the base substantially along the length. A bed is within the base and is configured to support a user, wherein the bed is substantially optically transparent that allows light therethrough. A hood is movably attached to the base and is movable between an open position and a closed position, wherein the hood comprises a second plurality of light emitting devices. The device includes first and second end caps attached to opposing ends of the base, wherein when the hood is in the closed position, a substantially enclosed chamber is formed between the base, the hood and the end caps. The device includes at least one airborne energy configured to be delivered into the enclosed chamber through at least one of the end caps. A controller is configured to control the type, intensity and duration of light emitted from the first and second plurality of light emitting devices and the at least one airborne energy to control an environment within the enclosed chamber. The device includes a control panel retained to the device or a graphic user interface remote from the device, wherein the control panel and/or the graphic user interface allows a user or provide to selected a predetermined therapeutic session or create a customized therapeutic session.

### BRIEF DESCRIPTION OF THE DRAWINGS

FIG. 1 is a front view of a therapeutic device.

FIG. 2 is isometric view an end cap attached to the therapeutic device.

FIG. 3 is another isometric view of the end cap attached to the therapeutic device.

FIG. 4 is a view of another embodiment of an end cap attached to the therapeutic device.

FIG. 5 is a schematic view of a control scheme for delivering energies within the therapeutic device.

### DETAILED DESCRIPTION

The present disclosure relates to a therapeutic device having an enclosed chamber in which a user can lay to receive light therapy and other natural therapies or treatments. The chamber includes a base and a hinged hood form a tubular space when the hood is closed. End caps are secured to the base at opposite ends that form a substantially enclosed capsule when the hood is closed. The end caps include inlets and optional outlets to allow the environment to be controlled to non-ambient conditions.

When the hood is closed, the affixed end-cap walls form a substantially enclosed chamber with fans and multiple portals allowing for entry, exhaust or interior re-circulation of selected energy deliveries into the enclosed chamber. The present disclosure allows for the controlled introduction of wellness energies that can be contained, circulated and/or recirculated within the enclosed chamber or pass through the enclosed chamber and optionally exhaust the wellness energies. The present disclosure also allows for the control of humidity and barometric pressure in aid in the absorption of the wellness energies.

In some embodiments. one or both end caps includes one or more optional fans, one or more ports for the introduction of wellness energies and one or more vents to exhaust air from the enclosed chamber. In some embodiments the fan(s) have one speed and in other embodiments the fans are variable speed.

The controlled dosage of the light therapy along with the modified environment through the addition of other energies within the enclosed chamber enhances, changes the outcome and/or has synergistic effects compared to a treatment with only light therapy. Exemplary, but non-limiting, additional energies that can be imparted into the enclosed chamber includes oxygen to enrich the environment, nebulized airborne liquid vitamins, nebulized airborne liquid trace minerals, nebulized liquid airborne liquid botanicals, nebulized airborne liquid cosmeceuticals, nebulized liquid pharmacologicals by themselves or combinations thereof. The introduction of the above energies, herein collectively referred to as “airborne energies” in controlled amounts by themselves or in combination with each other into the enclosed chamber increase the value of the treatment beyond that of only the light therapy treatment to the user.

Each of the airborne energies are independently controllable to customize the enclosed environment. Each airborne energy can be controlled for introduction, change in flow rate or concentration, pulsation, circulation, recirculation and or exhausting into the atmosphere with a control panel. The control panel can have preset routines for the introduction of the airborne energies or can be customized by the user or provider. The control panel can also be utilized to control the type and frequency of the light energy, the intensity of the light energy, the pulsation of the light energy and the time that the light energy is emitted. The control panel can be operated by the user or a provider of the enclosed capsule.

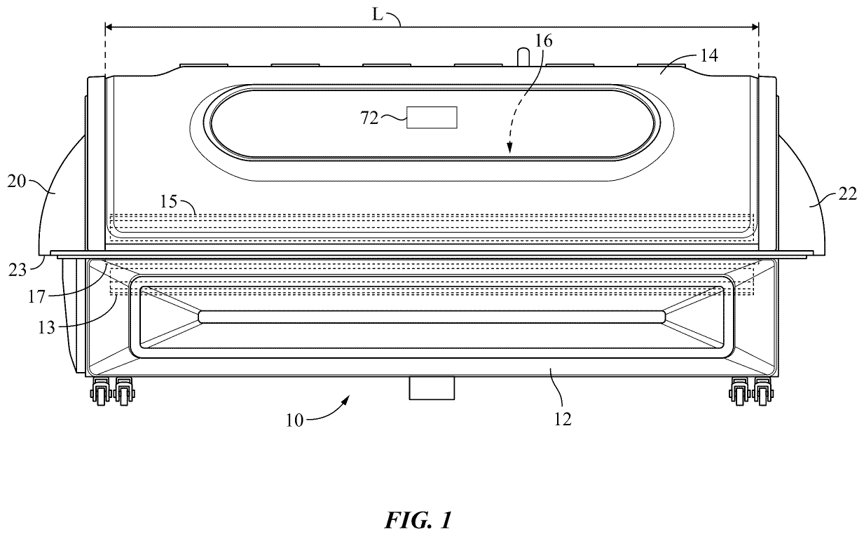

Referring to FIG. 1, a therapeutic device with an enclosed chamber is illustrated at 10. The device 10 includes a base 12 with a hood 14 hingedly attached thereto. A bed is supported in the base, where the bed can support electromagnetic emitters that emit electromagnetic pulses. When the hood 14 is pivoted about the hinges, a user gains access to an interior chamber 16. The base 12 and the hood 14 include a plurality of controllable light emitting device, such as LEDs and/or IR emitters, that substantially span a length L of both the base 12 and the hood 14.

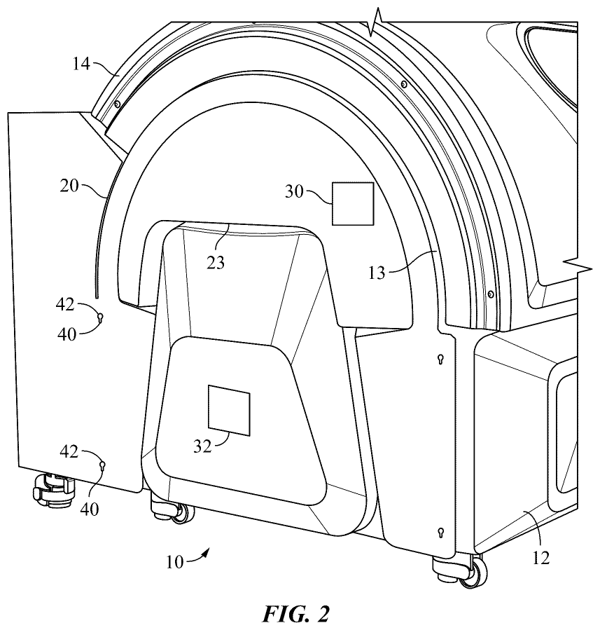

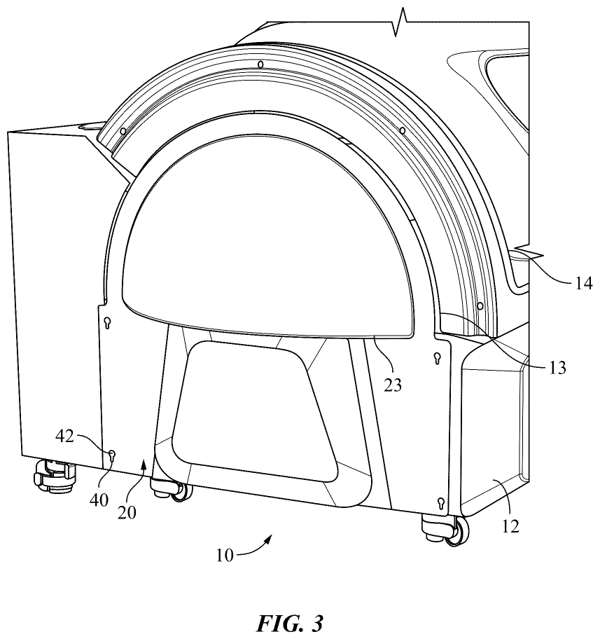

Referring to FIGS. 1-3, the device 10 includes end caps 20 and 22 that are attached to the base 12, such that the end caps are fixed relative to the base 12. The end caps 20 and 22 include an upper surface 24 that cooperates with an inner surface 13 of the hood 14 such that the interior chamber 16 is substantially enclosed.

The end caps 20 and 22 are attached to the base with bolts 40 secured within apertures in the base 12 where the heads of the bolts 40 pass through keyhole slots 42 in the end caps 20 and 22 and the constructed portion of the keyhole slots 42 such that the bold heads prevent the end caps 20 and 22 from disengaging the base 12. The bolts 20 are then tightened to frictionally secure the end caps 20 and 22 to the respective end of the base 12. While keyhole slots and bolts are described and illustrated herein, other attaching mechanisms are within the scope of the present disclosure, including but not limited to, screws, rivets, adhesives or cements and buckles.

Each end cap 20 and 22 includes a shelf 23 onto which the user can place personal belongings. Additionally, other energies can be placed in or around the shelf 23 including infrared energies and/or salt blocks to provide halo therapy and/or gemstones to provide additional energies.

Each end cap 20 and 22 can be is similarly constructed. As such, the end cap 20 will be described in detail and the description of end cap 20 applies to the end cap 22. The end cap 20 includes an air intake fan and a vent 32 with a controlled opening, such as a louver to control the air intake and exhaust into the interior chamber 16.

The end cap 20 includes a plurality of ports, each of the plurality of ports for one of the airborne energies. Alternatively, a single port can be utilized to provide access to the interior cavity 16 for each of the airborne energies.

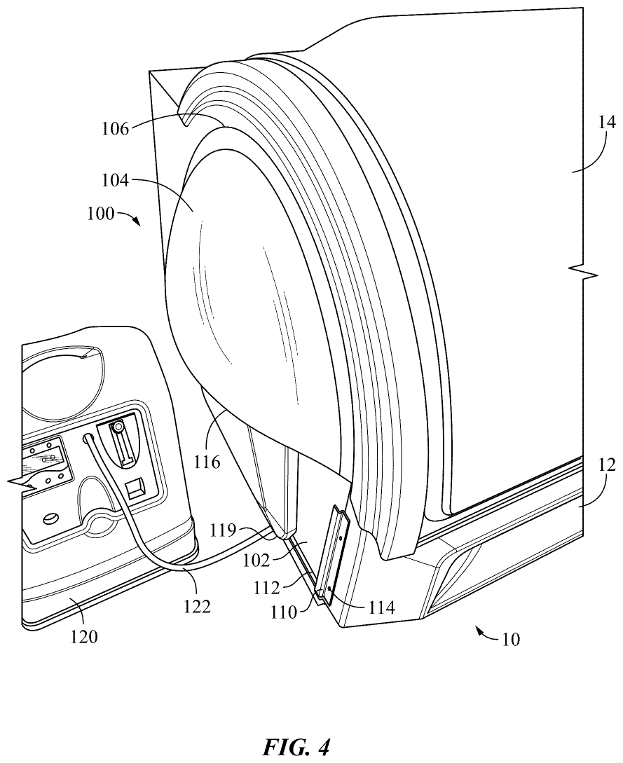

Referring to FIG. 4, another embodiment of an end cap is illustrated at 100. The end cap 100 is substantially a monolithic construction that is formed from a translucent polymer. The translucent polymer allows light to be emitted therethrough that allows a person spaced from the device to see a portion of the experience of the user.

A bottom portion 102 of the end cap 100 is retained within a mounting bracket 110 secured to the base 12. The mounting bracket 110 is “U” shaped, while only a portion is visible in FIG. 4. The mounting bracket 110includes a slot 112 into which the perimeter of the bottom portion 102 is positioned. Bolts and/or screws can be utilized to frictionally secure the bottom portion 102 between the mounting bracket 110 and the base 12.

The end cap 100 includes a top portion 104 that includes a top edge 106 that cooperates with an opening in the hood 14. As illustrated, the top edge 106 is arcuate. However, the top edge 106 can be any complementary configuration to that of the opening in the hood 14.

The end cap 100 includes a ledge 116 on which the person can place personal items. Alternatively, other energies can be placed on the ledge 116 including but not limited, IR emitters, salt blocks for halotherapy and gemstones to emit photons when heated.

A nebulizer 120 is illustrated in FIG. 4. The nebulizer 120 is configured to generate airborne energies and provide the airborne energies to the environment 16 through a hose 122 inserted into a port 119 in the end cap 100. The nebulizer 120 in FIG. 4 is generic in nature and can represent any nebulizer disclosed herein.

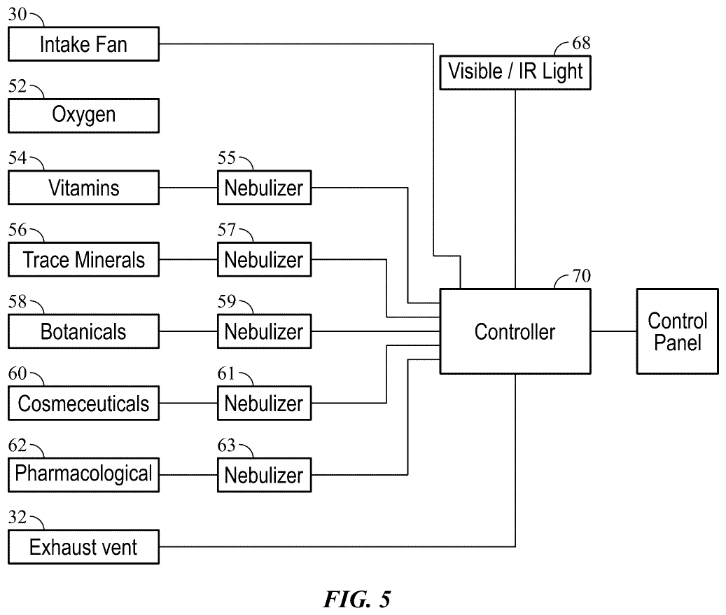

Referring to FIGS. 4 and 5, the supply or generation of the airborne energies including oxygen 52, vitamins 54, trace minerals 56, botanicals 58, cosmeceuticals 60 and pharmacologicals 62 can be within the end cap 20 or outside of the end cap. The vitamins 54, trace minerals 56, botanicals 58, cosmeceuticals 60 and pharmacologicals 62 have dedicated nebulizers 55, 57, 59, 61 and 63 for the respective airborne energy can be located in the end cap 20 or be spaced from the end cap 20. The nebulizers 55, 57, 59, 61 and 63 cause the energies to become airborne. Each of the nebulizers 55, 57, 59, 61 and 63 are in communication with a controller 70 that is configured to control the duration, dosage and time of the nebulization of the energy. The controller 70 also controls the oxygen used to enrich the environment in the internal chamber 16 along with the intake fan and vent.

The intake fan 30 pulls the nebulized airborne energy(s) into the interior chamber 16 and then circulates the airborne energy(s) therein where the speed of the optional fan 30 is controlled by the controller 30. The controller 70 is also configured to control the operation of the optional exhaust vent 32. Controlling the airborne energies 52, 54, 56, 58, 60 and 62, the visible and/or IR light 68 and the optional vent 32 allows the user to obtain results that are superior and different from that of light therapy alone.

The controller 70 is configured to control the optional fans 30 to adjust the air flow volume, speed, pulsation, flow direction and/or pressure of the airborne energies within the interior chamber 16. Additionally, the control 70 is configured to control the type of light emitted, the intensity, duration and pulsation of the selected light, whether in the visible spectrum or infrared spectrum.

The user or provider utilizes a control panel or graphic user interface 72 that is in communication with the controller to provide a customized environment within the interior chamber 16. The control panel or graphic user interface72 can be located on the device 10, on a computer spaced from the device or on an application downloaded on a mobile device. The control panel or graphic user interface 72 allows for the selection of a preprogramed therapy session with the light therapy and airborne energies or a customized therapy session with selected light therapy and/or airborne energies.

Although the present disclosure has been described with reference to preferred embodiments, workers skilled in the art will recognize that changes may be made in form and detail without departing from the spirit and scope of the disclosure.

1. A therapeutic device comprising:
a base having a length, the base comprising:
a first plurality of light emitting devices disposed in the base substantially along the length; and
a bed configured to support a user;
a hood movably attached to the base and movable between an open position and a closed position, wherein the hood comprises a second plurality of light emitting devices;
first and second end caps attached to opposing ends of the base, wherein when the hood is in the closed position, a substantially enclosed chamber is formed between the base, the hood and the end caps wherein at least one of the end caps houses a fan and an exhaust vent;
at least one airborne energy configured to be delivered into the enclosed chamber through at least one of the end caps; and
a controller configured to control a type, intensity and duration of light emitted from the first and second plurality of light emitting devices and a type and duration of the at least one airborne energy to control an environment within the enclosed chamber.

2. The therapeutic device of claim 1, wherein the at least one airborne energy comprises oxygen to enrich to the oxygen content within the enclosed chamber.

3. The therapeutic device of claim 1, wherein the least one airborne energy comprises nebulized airborne liquid vitamins, nebulized airborne liquid trace minerals, nebulized liquid airborne liquid botanicals, nebulized airborne liquid cosmeceuticals, nebulized liquid pharmacologicals by themselves or combinations thereof.

4. The therapeutic device of claim 1, and further comprising a fan, wherein the controller is configured control the fan to adjust the air flow volume, speed, pulsation, flow direction and/or pressure of the airborne energies within the interior chamber.

5. The therapeutic device of claim 4, and further comprising a vent, wherein the controller is configured to control a opening in the vent to allow a selected amount of air to exhaust from the enclosed chamber.

6. The therapeutic device of claim 3, wherein a source of the at least one airborne energy is spaced from the first end cap.

7. The therapeutic device of claim 3, wherein a source of the at least one airborne energy is located with the first end cap.

8. The therapeutic device of claim 1, wherein at least the first end cap includes a shelf.

9. The therapeutic device of claim 1, wherein the shelf is configured to support other energies comprising infrared energy, gemstones, salt and combinations thereof.

10. The therapeutic device of claim 1 and further comprising a control panel on the hood, the control panel in communication with the controller, wherein the control panel is configured to allow a user or a provider to select a therapeutic regimen.

11. The therapeutic device of claim 1 and further comprising a graphic user interface in communication with the controller, wherein the graphic user interface is configured to allow a user or a provider to select a therapeutic regimen.

12. The therapeutic device of claim 11, wherein the graphic user interface is located on a computer screen or a hand held electronic device.

13. The therapeutic device of claim 1, wherein the first and second plurality of light emitting devices comprise light emitting diodes.

14. The therapeutic device of claim 1, wherein a barometric pressure within the enclosed chamber is controllable.

15. The device of claim 1, wherein a relative humidity within the enclosed chamber is controllable.

16. A therapeutic device comprising:
a base having a length, the base comprising:
a first plurality of light emitting devices disposed in the base substantially along the length; and
a bed configured to support a user;
a hood movably attached to the base and movable between an open position and a closed position, wherein the hood comprises a second plurality of light emitting devices;
first and second end caps attached to opposing ends of the base, wherein when the hood is in the closed position, a substantially enclosed chamber is formed between the base, the hood and the end caps;
at least one airborne energy configured to be delivered into the enclosed chamber through at least one of the end caps;
a controller configured to control the type, intensity and duration of light emitted from the first and second plurality of light emitting devices and a type and duration of at least one airborne energy to control an environment within the enclosed chamber; and
a control panel retained to the device or a graphic user interface remote from the device, wherein the control panel and/or the graphic user interface allows a user or provide to selected a predetermined therapeutic session or create a customized therapeutic session.

17. The therapeutic device of claim 16, wherein the at least one airborne energy comprises oxygen to enrich to the oxygen content within the enclosed chamber.

18. The therapeutic device of claim 16, wherein the least one airborne energy comprises nebulized airborne liquid vitamins, nebulized airborne liquid trace minerals, nebulized liquid airborne liquid botanicals, nebulized airborne liquid cosmeceuticals, nebulized liquid pharmacologicals by themselves or combinations thereof.

19. The therapeutic device of claim 16, and further comprising a fan, wherein the controller is configured control the fan to adjust the air flow volume, speed, pulsation, flow direction and/or pressure of the airborne energies within the interior chamber.

20. The therapeutic device of claim 19, and further comprising a vent, wherein the controller is configured to control an opening in the vent to allow a selected amount of air to exhaust from the enclosed chamber.
